(19) United States
(12) Patent Application Publication
(10) Pub. No.: US 2002/0082669 A1
(43) Pub. Date: Jun. 27, 2002

(54) Apparatus for irradiating blood with UV energy

(76) Inventor: J. Leonard Nitsch, Syracuse, NY (US)

Correspondence Address: WALL MARJAMA & BILINSKI, 101 SOUTH SALINA STREET, SUITE 400, SYRACUSE, NY, 13202, US

(21) Appl. No.: 09/746,248

(22) Filed: Dec. 21, 2000

(51) Int. Cl. A61M 37/00

(52) U.S. Cl. 607/100

(57) ABSTRACT

Apparatus for treating blood that includes a source of ultraviolet radiation arranged to irradiate a target region. A cuvette fabricated of a single tube of quartz that contains a series of parallel runs that are connected by 180° bends so that the runs and bend lie in a common plate within the target region. An aspirator is connected to the inlet of the cuvette and a vacuum bottle or pump is connected to the outlet of the cuvette. The inside diameter of the cuvette is sized so that a uniform flow of blood moves through the cuvette and the blood is well mixed as it moves through the bends.

### FIELD OF THE INVENTION

This invention relates generally to ultraviolet blood irradiation, and in particular to improved apparatus for treating blood with ultra-violet radiation.

### BACKGROUND OF THE INVENTION

Over the last fifty years or more, ultraviolet irradiation of blood has been used to treat a widening number of diseases. It has been found that the intravenous administration of ultraviolet energy to a patient in controlled dosages has many advantages when compared to the external administration of this type of energy. Absorption of the ultraviolet energy by blood is predictable as to both the rate of absorption and the amount of energy that is absorbed. Typically, blood is withdrawn from the patient and passed through a cuvette made of quartz wherein it is exposed to ultraviolet energy at a desired intensity level and for a given duration to produce the correct photochemical reaction. Overexposure or underexposure of the blood can produce little or no beneficial results and in the case of overexposure, can be, under certain conditions, harmful to the patient. An optimum dosage, on the other hand, can produce truly therapeutic results involving a wide number of diseases.

Apparatus has been devised for treating blood with ultraviolet radiation which affords a certain amount of control over the exposure process. Heretofore, most of these machines utilize a flat quartz cuvette having a shape similar to that shown in FIG. 3. The prior art cuvette 10, as illustrated, contains a restrictive entrance port 12 and a correspondingly restrictive outlet port 13, both having a cylindrical cross section which are removably connected to an inlet tube 15 and an outlet tube 16. The two ports are centered along the axis 18 of the cuvette and, as can be seen, the cross sectional area of the cuvette is considerably greater than that of the aligned ports. During the blood irradiation process a main flow stream 20 is established within the cuvette that extends in a straight line along the axis of the cuvette between the two ports. Vortices 21 are generated in the cuvette to either side of the main stream. Each vortex that develops within the cuvette can adversely effect the uniformity of flow through the cuvette and thus the controllability of the radiation dosage absorbed by the blood. Blood traveling in the central stream moves more rapidly through the radiation zone than blood trapped in the vortices. Accordingly, a portion of the blood may become underexposed while another portion overexposed which, in turn, can adversely effect the results of the treatment being administered to the patient. Similarly, under certain conditions, stagnant regions can further develop in certain areas within the cuvette causing blood in these regions to begin to clot again adversely effecting the results of the treatment.

### SUMMARY OF THE INVENTION

It is therefore an object of the present invention to improve the effectiveness of ultraviolet radiation of blood for therapeutic purposes.

It is a further object of the present invention to more closely control the irradiation blood as it passes through an ultraviolet radiation zone.

A still further object of the present invention is to provide an improved cuvette for use in apparatus for irradiating blood with ultraviolet energy.

Another object of the present invention is to provide a mixing action so that a uniform flow of blood is established within the ultraviolet radiation zone.

These and other objects of the present invention are attained by means of apparatus for irradiating blood with ultraviolet energy that includes a source of ultraviolet energy arranged to irradiate a target region. A cuvette fabricated of a length of cylindrical tubing is made of material that transmits ultraviolet energy. The tubing is formed into a serpentine shape having parallel runs that are connected by 180° bends, all of which lie in a common plane within the target zone of the energy source. One end of the cuvette is connected to an aspirator (needle) for withdrawing blood from a patient and the other end to a vacuum source so that blood from the patient is drawn through the cuvette where it is irradiated with ultraviolet energy. The inside diameter of the cuvette is uniform throughout so that a uniform flow of blood moves through the radiation zone. A well mixed flow is created in the bend regions which thoroughly mixes the blood that is in transit.

### BRIEF DESCRIPTION OF THE DRAWING

For a further understanding of these and objects of the invention, reference will be made to the following detailed description of the invention which is to be read in connection with the accompanying drawing, wherein:

FIG. 1 is a schematic representation of apparatus embodying the teachings of the present invention;

FIG. 2 is a partial perspective view showing the irradiating region of the apparatus illustrated in FIG. 1;

FIG. 3 is a perspective view of a cuvette found in the prior art for conveying blood through an ultraviolet irradiating region of a blood treatment device;

FIG. 4 is a perspective view showing another embodiment of a cuvette suitable for use in the present invention;

FIG. 5 is an end view taken along lines 5-5 in FIG. 4;

FIG. 6 is a further embodiment of the present invention; and

FIG. 7 is a sectional view taken along lines 7-7 in FIG. 6.

### DETAILED DESCRIPTION OF THE INVENTION

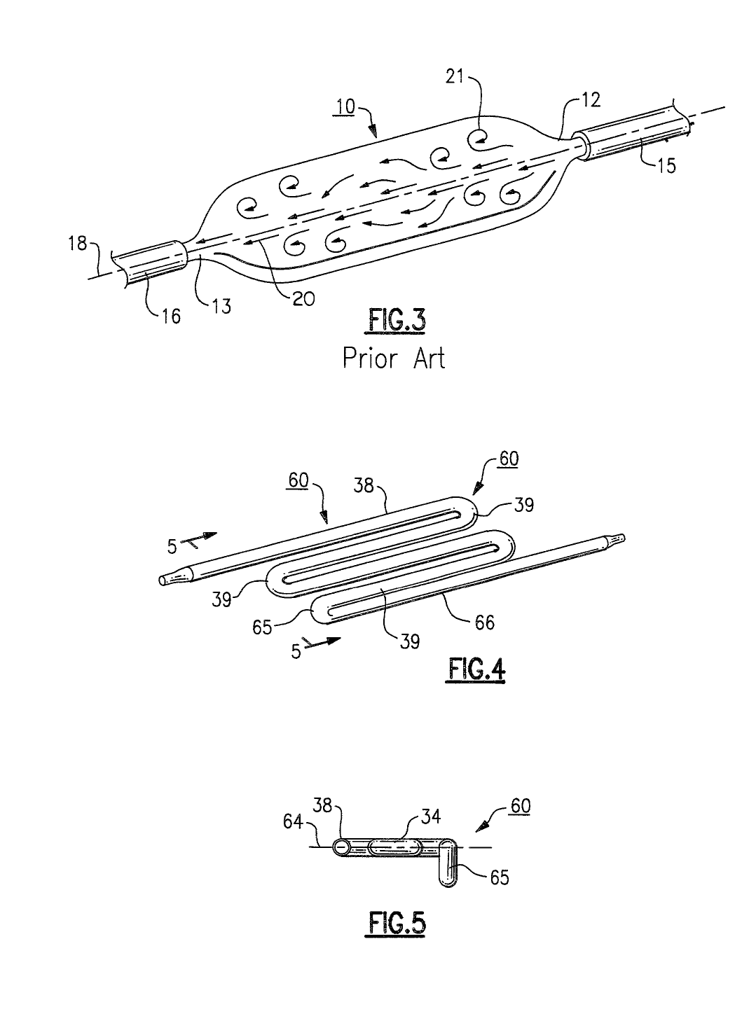

Referring once again to FIG. 3, as explained above, the main flow stream 20 conducted through the flat cuvette 10 moves in a straight reasonably undisturbed line between the inlet and outlet ports of the device. It is known that hemoglobin pigments in blood are the part of the blood that strongly absorbs ultraviolet energy. The remaining part of the blood, however, is generally opaque to ultraviolet energy. As a result, the blood which is moving in the generally quiescent flow stream 20 through the flat cuvette 10 will not uniformly absorb the ultraviolet energy to which it is exposed. The UV-absorbing hemoglobin pigments on the lower part of the flow stream that are shielded from the energy source by the stream, will not absorb the energy at the same rate as that in the upper part of the stream. As a result, the blood in the flow stream cannot uniformly absorb the ultraviolet energy. By the same token, the blood that moves to either side of the main stream can become trapped in the vortices. Although the blood is well mixed in this area, its movement across the cuvette is relatively slow. As a consequence, the blood in this area can become overexposed. The net result of this is that the irradiation of the blood cannot be accurately controlled in this type of device and the therapeutic value of the treatment is greatly reduced.

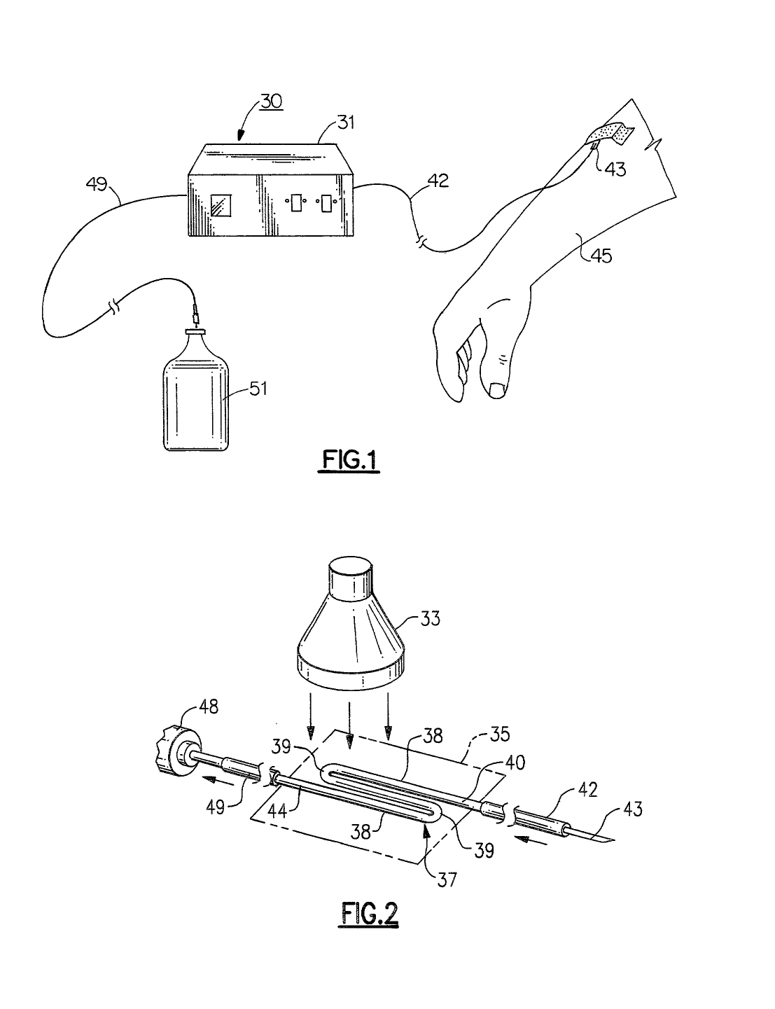

Turning now to FIGS. 1 and 2, there is shown apparatus 30 for irradiating blood that embodies the teachings of the present invention. The apparatus includes a housing 31 containing a high intensity quartz-mercury ultraviolet lamp 33 that has a relatively high UV-B output. The lamp is arranged to focus UV-radiation within a given region 35 within the housing. A serpentine shaped cuvette, generally referenced 37, is mounted within the irradiated region. The cuvette preferably is fabricated of a single length of quartz tubing that is bent into three parallel runs 38-38 that are connected by 180° bends. Although quartz tubing is herein disclosed, any material that is transparent to ultraviolet radiation can be used in the practice of the present invention. The entrance end 40 of the cuvette passes out of the radiation region and is connected by an inlet line 42 to a needle 43 for aspirating blood from a patient 45. The exit end 44 of the cuvette also passes out of the radiation region and is connected to a peristaltic pump 48 by means of an outlet line 49. The pump is contained within the housing and is adapted to draw blood from the patient and pass the blood through the cuvette at a controlled rate of flow. The blood that has been irradiated is collected in a bottle 51 for reinfusion back into the patient when the irradiation process is completed.

The inside diameter of the cuvette tube is uniform throughout the entire length of the cuvette. Accordingly, the rate of flow through the cuvette cannot be adversely effected by changes in the area of the flow path. The linear tube runs 38 are placed in close proximity with each other so that the tube bends 39 are relatively tight. The flow moving through the bends therefore becomes well agitated to insure thorough mixing of the blood in transit. As a result, the UV-absorbing hemoglobin pigments in the blood flow are uniformly irradiated as the blood moves through the cuvette.

Although a peristaltic pump may be used to draw blood from the patient, the pump may be eliminated and a vacuum bottle of the type that is well known in this art used in its stead. Once the vacuum bottle is filled with a desired amount of blood, the vacuum is released and the bottle then used to gravity feed the blood back into the patient. Accordingly, the pump can be bypassed from housing 31 and the collection bottle 51 replaced with a vacuum bottle without departing from the teachings of the present invention. Similarly, the cuvette may have more or less parallel runs without departing from the teachings of the invention provided all the runs and bends lie within the irradiated area of the lamp.

Turning now to FIGS. 4 and 5, there is shown a second cuvette 60 that is suitable for use in the practice of the present invention. Here again, the cuvette is fabricated from a single length of quartz tubing having a series of straight runs 38-38 that are connected by tube bends 39-39, all of which lie in a common plane. In this embodiment of the invention, an equal number of runs are situated in a common plane 64 (FIG. 5). The cuvette is further equipped with an offset return run 66 that is in parallel alignment with the last run in the series, however, the return run, as illustrated in FIG. 5 is positioned beneath the plane 64 in which the other runs 38 are situated. Although the return run is shown located below the plane 64, it should be obvious to one skilled in the art that the return run can be similarly positioned above the plane. A 180° bend connects the return run with the last run in the series situated in the plane. As should now be evident, the return run, when used in conjunction with an even number of coplanar runs, allows the entrance to the cuvette to be located on one side of the irradiated region and the exit to be located on the opposite side of the irradiated region, which may have advantages in certain applications.

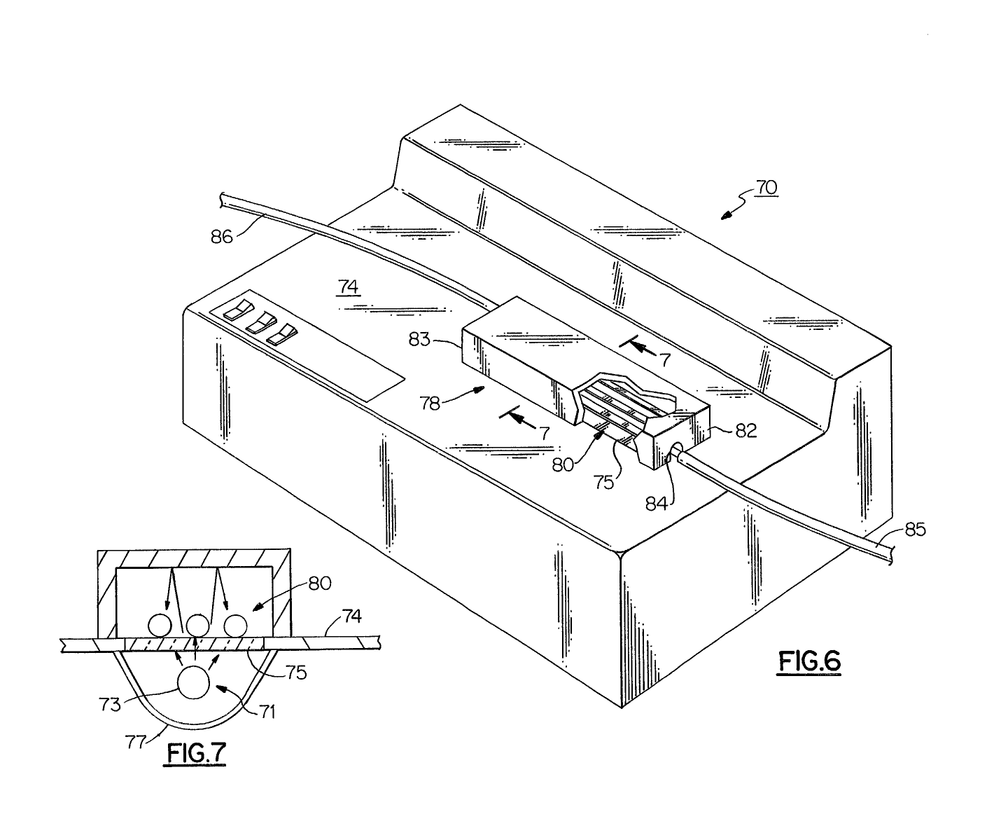

Turning now to FIGS. 6 and 7 there is shown a further embodiment of the invention that includes a light box generally referenced 70 which contains an ultraviolet light source 71. The top wall 74 of the machine is a flat platen 74 that contains a rectangular quartz window 75. The light source is an elongated lamp 73 that is centered along the longitudinal axis of the window and extends along its entire length. The lamp is contained within a light tight enclosure 77. Preferably, the interior surface of the enclosure is coated with a reflective material and the enclosure is contoured to distribute the reflected energy uniformly over the bottom light entrance face of the quartz window. Although not shown, the light box further contains an electrical power supply and circuitry for controlling the operation of the lamp.

A generally rectangular shaped housing 78 is mounted upon the platen over the window. The housing frames the entire window surface and has a height such that a serpentine shaped cuvette 80 of the type described above, can be supported therein in contact against the top surface of the window 75. The opposed end walls 82, 83 of the housing are provided with openings 84 through which the entrance and exit runs of the cuvette can enter and leave the housing. As explained above, the entrance and exit ends of the cuvette are connected to inlet and outlet lines 85 and 86, respectively, so that a supply of blood under treatment can flow uniformly through the cuvette. In this embodiment of the invention the bends and parallel runs of the cuvette are arranged so that they lie inside the boundaries of the window and are thus exposed to radiation from the ultraviolet lamp.

The housing is preferably fabricated of a highly reflective material such as polished aluminum or stainless steel so that light energy striking the interior surfaces of the housing is reflected back toward the interior of the housing. Alternatively, the inner surfaces of the housing may be coated with a highly reflective material for producing the same results.

As best illustrated in FIG. 7, some of the ultraviolet radiation from the light source or that reflected from the lamp enclosure will enter the cuvette and be absorbed by the in process blood flow. Some of this energy, on the other hand, will pass upwardly between the parallel runs of the cuvette and strike the inner wall surfaces of the housing and be reflected back toward the cuvette. As a result, most of the energy from the lamp is utilized in the treatment process and the energy is more evenly distributed over the flow of blood moving through the cuvette.

While the present invention has been particularly shown and described with reference to the preferred mode as illustrated in the drawing, it will be understood by one skilled in the art that various changes in detail may be effected therein without departing from the spirit and scope of the invention as defined by the claims.

We claim:

1. Apparatus for treating blood that includes:
an energy source for focusing ultraviolet radiation within a target region,
a cuvette fabricated of a cylindrical tube of material that is transparent to ultraviolet radiation and having a uniform inside diameter,
said cuvette containing a series of parallel runs lying in a common plane within the target region, said runs being connected by 180° bends, all of which lie within the target region, said cuvette having an inlet end and an outlet end,
aspirator means for withdrawing blood from a patient that is connected to the inlet of said cuvette, and
a vacuum means that is connected to the outlet of said cuvette for drawing blood through said cuvette.

2. The apparatus of claim 1 wherein said cuvette contains an odd number of runs.

3. The apparatus of claim 1 wherein the inside diameter of the cuvette is sized so that a flow of blood is produced in said bends whereby blood in the flow passing through the cuvette is well mixed.

4. The apparatus of claim 1 wherein said vacuum means is a vacuum bottle connected to the outlet of said cuvette.

5. The apparatus of claim 1 wherein said vacuum means is a peristaltic pump connected to the outlet of said cuvette.

6. The apparatus of claim 1 wherein the inlet and outlet of said cuvette lie outside of said target region.

7. The apparatus of claim 6 wherein the inlet and outlet of said cuvette are extensions of two end runs of said cuvette.

8. The apparatus of claim 1 wherein said cuvette is formed of quartz crystal.

9. The apparatus of claim 1 wherein said cuvette includes a return run that is parallely aligned above or below the last run in said common plane with regard to the direction of flow through said cuvette, said return run being connected to said last run by a 180° bend.

10. Apparatus for the treatment of blood that includes
a flat platen that contains a rectangular shaped window fabricated of a material that is transparent to ultraviolet radiation;
an elongated ultraviolet lamp mounted beneath said window for directing ultraviolet radiation through said window;
a serpentine shaped cuvette containing a series of parallel runs that are connected by 180° bends seated upon the top of said window;
means for passing a flow of blood through said cuvette whereby said blood is treated by said ultraviolet radiation; and
a housing mounted upon said platen over said window so that said window is fully enclosed within the housing, the interior surfaces of said housing being reflective to ultraviolet energy so that ultraviolet energy striking the interior surfaces is reflected back into the housing.

11. The apparatus of claim 10 wherein said housing contains opposed end walls, each having an opening for allowing inlet and outlet runs of the cuvette to pass therethrough.

12. The apparatus of claim 10 wherein said lamp is contained within an enclosure, the interior of which is reflective to ultraviolet light.

13. The apparatus of claim 12 wherein said enclosure is contoured to direct reflective light at said window and uniformly distribute said light over the surface of the window.

14. The apparatus of claim 10 wherein the runs and the bends of said cuvette lie inside the boundaries of said window.
